(19) United States
(12) Patent Application Publication
(10) Pub. No.: US 2026/0306430 A1
(43) Pub. Date: Oct. 8, 2026

(54) Compositions, Kits, and Methods Comprising Bioidentical D BHB With Optional Vitamin B6 for Neurological Wellness, Mood Stability, and Medication Taper Support

(71) Applicant: Global Ketone Limited, Hong Kong SAR (HK)

(72) Inventors: Heidi Heather Hynes, Seattle, WA (US); James Schroeder, Seattle, WA (US)

(21) Appl. No.: 19/697,777

(22) Filed: Jun. 4, 2026

Related U.S. Application Data
(63) Continuation-in-part of application No. 17/002,691, filed on Aug. 25, 2020.
(60) Provisional application No. 63/013,340, filed on Apr. 21, 2020.

(51) Int. Cl. A61K 31/19; A61B 5/16; A61K 31/205; A61K 31/4188; A61K 31/4415; A61K 31/51; A61K 31/525; A61K 31/714; G01N 33/53; G01N 33/543; G01N 33/64; G01N 33/66

(52) U.S. Cl. CPC A61K 31/19; A61K 31/205; A61K 31/4188; A61K 31/4415; A61K 31/51; A61K 31/525; A61K 31/714; G01N 33/5306; G01N 33/54387; G01N 33/64; G01N 33/66; A61B 5/165; G01N 2800/52

(57) ABSTRACT

Compositions, kits, and methods are disclosed for nutritional neurometabolic support and improved gut health using bioidentical D-beta-hydroxybutyric acid (D-BHB) to promote neurological wellness, mood stability and adaptive brain function during everyday life and during changes in neurologically acting medication regimens. The compositions comprise a salt-free, ester-free, alcohol-free D-BHB acid formulation optionally configured with vitamin B to raise circulating ketone levels, supply a preferred brain energy substrate, and support intestinal barrier integrity by improving tight junction function and reducing paracellular leakiness, thereby influencing gut-brain axis signaling. The compositions optionally incorporate B vitamins with vitamin B6 being a preferred embodiment to complement bioidentical D-BHB by supporting neurotransmitter synthesis and related gut-brain pathways. Kit (701) provides unit dose containers of bioidentical D-BHB, with or without B-vitamin supplementation and optional tapered pharmaceutical doses, together with optional software or AI-based tools configured to capture diet and mood data via smartphone and to generate guidance for nutritional and medication-taper support.

### RELATED APPLICATIONS

This application is a continuation-in-part of U.S. application Ser. No. 17/002,691, filed Aug. 25, 2020, which claims the benefit of U.S. Provisional Application Ser. No. 63/013,340, filed Apr. 21, 2020, the entire contents of each of which are incorporated by reference herein.

### BACKGROUND OF THE INVENTION

Neurologically acting pharmaceutical agents are widely used to treat a variety of conditions affecting the central nervous system and can be associated in some individuals with changes in mood, cognition, sleep, and overall neurological comfort. Use of such medications may also coincide with gastrointestinal (GI) changes, including altered gut function and impaired GI barrier integrity, which are increasingly recognized as relevant to brain and mood regulation through the gut-brain axis.

In everyday practice, clinicians primarily rely on medication labeling, clinical guidelines, and visit-based counseling, while individuals frequently turn to general nutritional products and informal lifestyle measures to support wellbeing as these agents are used, adjusted, or tapered. These consumer- and clinic-side approaches can be variable in content and structure, and are not typically organized around coordinated support for neurometabolic function, gut health, and day-to-day adaptation to changes in neurologically acting medication regimens.

### FIELD OF THE INVENTION

The present invention relates to compositions, kits, systems, and methods in the fields of gut-brain health, nutritional neuroscience and neurometabolic support. More specifically, the invention concerns the use of bioidentical D-beta-hydroxybutyric acid (D-BHB), optionally in combination with one or more B vitamins such as vitamin B6, to support brain energy metabolism, intestinal barrier integrity, and mood and cognitive stability in individuals using, adjusting, or tapering neurologically acting medications. The invention further relates to kits and software-assisted guidance systems that combine such nutritional compositions with optional digital tools configured to capture diet and mood information and to provide user-specific guidance for nutritional and medication-taper support.

### SUMMARY OF THE INVENTION

The present invention provides nutritional compositions, kits, systems, and methods that use bioidentical D-beta-hydroxybutyric acid (D-BHB) to support gut-brain health, neurometabolic function, and mood and cognitive stability in individuals who are using, adjusting, or tapering neurologically acting medications. In various embodiments, the compositions comprise salt-free, ester-free, alcohol-free bioidentical D-BHB acid formulations configured to elevate circulating ketone levels, supply a preferred brain energy substrate, and support intestinal barrier integrity by improving tight junction function and reducing paracellular leakiness, thereby modulating gut-brain axis signaling, for example via vagal pathways.

In certain embodiments, the compositions further comprise one or more B vitamins, with vitamin B6 as a preferred embodiment, to complement D-BHB by supporting neurotransmitter synthesis, homocysteine metabolism, and gut barrier integrity in individuals with increased need or deficiency. The invention includes both D-BHB-only and D-BHB-plus-B-vitamin embodiments, allowing the ketone component to serve as the primary neurometabolic agent while B-vitamin co-administration provides an optional, preferred enhancement.

Bioidentical D-BetaHydroxybutyric Acid and optional vitamin B6 supports gut-brain health, brain network stability and GABA-glutamate balance. In specific embodiments the claimed compounds improve seizure threshold during epilepsy and more broadly support withdrawal-related tapering. In one aspect, the claimed invention thus provides a composition comprising bioidentical D-BHB acid, optionally in combination with at least one B vitamin, such as vitamin B6, formulated for oral administration as a liquid, powder, capsule, or other ingestible dosage form. Additional foreseeable embodiments incorporate pre-measured pharmaceutical agent taper doses In another aspect, the invention provides methods of using such compositions to promote gut-brain health, neurometabolic resilience, and mood and cognitive stability in subjects exposed to centrally acting medications, including but not limited to antiepileptic drugs, antidepressants, and anxiolytics to allow for cessation without (or greatly reduced) unwanted side effects.

The invention further provides kits that include unit-dose containers of bioidentical D-BHB, with or without B-vitamin supplementation, and printed or electronic instructions for use in supporting neurological wellness, gut health, and adaptive brain function during periods of stable use, adjustment, or tapering of neurologically acting medications. In some embodiments, the kits are integrated with software-assisted guidance systems comprising optional digital tools, such as smartphone applications or web-based interfaces, configured to capture diet, medication, biomarkers, in home testing such as ketone and glucose blood test strips along with external testing including insulin, Zonulin, Aldosterone, HBA1c, and mood data and to provide user-specific guidance for nutritional support and medication-taper planning, ideally in collaboration with a clinician. In particular embodiments, the invention is applied to subjects receiving antiepileptic drugs (AEDs), such as levetiracetam, and provides nutritional support for behavioral and mood manifestations that may arise during ongoing AED therapy or during dose adjustment and tapering. In these embodiments, the compositions can be used as neurometabolic adjuvants to help support mood stability, cognitive clarity, and seizure-threshold resilience while standard pharmacologic regimens are maintained, modified, or reduced under medical supervision.

In further aspects, the invention provides methods of supporting dose reduction or discontinuation of neurologically acting medications, including Selective serotonin reuptake inhibitors (SSRIs), Attention-Deficit/Hyperactivity Disorder (ADHD) medications, antiepileptic drugs and Benzodiazepines alone or in combination in a subject by administering a composition comprising bioidentical D-BHB, optionally together with vitamin B6, according to a metabolic support protocol that includes a pre-tapering phase, a tapering phase concurrent with stepwise dose reduction, and a post-tapering maintenance phase. In these methods, D-BHB provides ketone-based brain energy and neurometabolic support, while vitamin B6, when present, further supports GABA and monoamine neurotransmitter pathways and related mechanisms.

In certain mechanistic embodiments, the invention exploits the convergence of D-BHB and vitamin B6 on GABAergic neurotransmission and neuroinflammatory pathways. D-BHB can upregulate GAD1 expression and preserve glutamate substrate for GABA synthesis via SIRT4-mediated modulation of glutamate dehydrogenase, while vitamin B6 (for example in the form of pyridoxal-5-phosphate) serves as an obligate cofactor converting glutamate decarboxylase from its inactive apoenzyme form to its active holoenzyme form. In combination, these mechanisms can increase both the quantity of available GAD enzyme and the fraction that is catalytically active, enhancing GABA synthesis capacity.

In additional mechanistic embodiments, D-BHB and vitamin B6 each contribute to the modulation of neuroinflammation, including through effects on pathways such as NLRP3 inflammasome signaling and related cytokine cascades, thereby providing further support for neural network stability during exposure to or withdrawal from neurologically acting medications.

In yet another aspect, the invention provides systems and methods in which nutritional administration of bioidentical D-BHB, with or without B-vitamin co-supplementation, is coordinated with software-assisted monitoring and guidance. Data such as dietary intake, medication dosing, blood or breath ketone measurements, glucose values, and mood assessments may be captured via a user device and processed by an AI-assisted or rule-based engine to generate individualized recommendations on timing and magnitude of D-BHB dosing, dietary adjustments, and coordination with medically supervised medication-taper schedules.

### BRIEF DESCRIPTION OF THE DRAWINGS

The accompanying drawings, which are incorporated in and constitute a part of this specification, illustrate preferred embodiments of the invention and, together with the description, serve to explain the principles of the invention.

FIG. 1 is a schematic illustration of normal intestinal barrier integrity compared with increased intestinal permeability (“leaky gut”) in the human gastrointestinal tract.

FIG. 2 schematically illustrates restoration of intestinal tight junctions by D-β-hydroxybutyric acid.

FIG. 3 schematically illustrates improvement of intestinal tight junction integrity and reduction of paracellular leakiness in response to administration of D-β-hydroxybutyric acid in combination with vitamin B6.

FIG. 4 depicts a graphical illustration of negative physiological impact of pharmaceuticals on the human brain.

FIG. 5 is a schematic illustration of a cortical neural network under chronic exposure to pharmaceutically administered (exogenous) neurotransmitters, showing reduced endogenous neurotransmitter signaling and decreased intrinsic neuronal firing.

FIG. 6 is a schematic illustration of a cortical neural network receiving ketogenic metabolic support from D-β-hydroxybutyric acid, showing balanced endogenous neurotransmission, controlled exogenous neurotransmitter input, and improved network stability.

FIG. 7 illustrates an illustrative embodiment of a ketosis enhanced mood stabilization and pharmaceutical deprescription kit (701).

FIG. 8 is a schematic flow diagram of a method for administering D-beta-hydroxybutyric acid, optionally with B vitamins, together with optional diet, pharmaceutical, ketone, glucose, and mood data capture steps to support neurological stabilization during psychotropic drug use and deprescription.

### DETAILED DESCRIPTION OF PREFERRED EMBODIMENTS

The following detailed description sets forth specific preferred embodiments of the invention with sufficient detail to enable a person of ordinary skill in the art to practice the invention. This detailed description is not intended to limit the scope of the invention as defined in the appended claims.

### Definitions

“As used herein, the following terms have the meanings set forth below, unless the context clearly indicates otherwise.”

### D-beta-hydroxybutyric Acid, D-bhb, or Bioidentical D-bhb

“D-beta-hydroxybutyric acid,” “D-BHB,” or “bioidentical D-BHB” refers to the D-enantiomer (R-enantiomer) of beta-hydroxybutyric acid, corresponding to the ketone body produced endogenously in humans during fasting or ketogenic states. As used herein, “bioidentical D-BHB” denotes D-beta-hydroxybutyric acid in entirely enantiomerically pure form, containing the D-enantiomer and no L-beta-hydroxybutyric acid (S-enantiomer), such that the L-enantiomer is not present. Unless otherwise specified, preferred embodiments employ the free acid form of bioidentical D-BHB, optionally together with acceptable excipients, while alternative embodiments may employ pharmaceutically acceptable salts, esters, or precursor forms that are metabolized to D-BHB upon administration.

### B Vitamin and Vitamin B6

“B vitamin” refers generally to any vitamin of the B-complex group, including without limitation vitamins B1, B2, B3, B5, B6, B7, B9, and B12, and combinations thereof. “Vitamin B6” refers to vitamin B6 in any physiologically acceptable form, including pyridoxine, pyridoxal, pyridoxamine, and, in particular, pyridoxal-5-phosphate (P5P or PLP), which is the biologically active coenzyme form and constitutes a preferred embodiment in certain compositions and methods of the present invention.

### Neurologically Acting Medication

“Neurologically acting medication” refers to any pharmacological agent that exerts a primary or substantial effect on the central nervous system, including without limitation antiepileptic drugs (AEDs), antidepressants, anxiolytics, antipsychotics, mood stabilizers, Selective serotonin reuptake inhibitors (SSRIs), Attention-Deficit/Hyperactivity Disorder (ADHD) medications, Benzodiazepines and other centrally acting psychotropic or neuromodulatory agents alone or in combination.

### Gut-Brain Axis

“Gut-brain axis” refers to the bidirectional network of communication pathways between the gastrointestinal tract and the central nervous system, including but not limited to neural pathways (such as vagal signaling), endocrine pathways, immune and cytokine signaling, and microbial or metabolite-mediated mechanisms, through which changes in intestinal barrier integrity, gut function, or microbiota composition can influence mood, cognition, and neurological function.

### Intestinal Barrier Integrity

“Intestinal barrier integrity” refers to the structural and functional state of the gastrointestinal mucosal barrier, including epithelial tight junctions and associated cellular and molecular components, that together regulate paracellular permeability and restrict inappropriate passage of luminal contents into underlying tissue. An “improvement in intestinal barrier integrity” or “reduced paracellular leakiness” refers to a change consistent with tighter junctions, reduced marker permeability, or restoration toward a healthy barrier state.

### Software-assisted Guidance System

“Software-assisted guidance system” refers to any combination of software, algorithms, and user interfaces, optionally hosted on a smartphone, tablet, computer, or web-based platform, configured to receive data related to at least one of dietary intake, medication dosing, biomarker measurements, or mood assessments and to output guidance, recommendations, or prompts relating to nutritional support and/or coordination with medically supervised changes in neurologically acting medications.

FIG. 1 illustrates a portion of a human gastrointestinal tract 10, with an enlarged view of the intestinal epithelium showing, on one side, a normal epithelial barrier with intact villi and tight junctions 12 and, on the other side, an epithelial barrier exhibiting increased intestinal permeability 13 (“leaky gut”). In the normal region, epithelial cells form continuous tight junctions and well-formed villi that limit paracellular passage of luminal contents into underlying tissue, representing preserved intestinal barrier integrity. In the leaky-gut region, blunted villi and disrupted junctions allow luminal particles 14, 15 to pass between adjacent epithelial cells into the lamina propria, representing increased paracellular leakiness. Such impairment of intestinal barrier function has been associated with conditions including celiac disease and inflammatory bowel disease and can be influenced in susceptible individuals by factors such as diets high in ultra-processed foods, chronic stress, and long-term use of certain neurologically acting medications. Increased intestinal permeability 13 may contribute, via systemic immune activation and gut-brain axis signaling, to changes in mood, cognition, and overall neurological comfort in certain subjects, providing context for the gut-brain support addressed by the compositions and methods of the present invention.

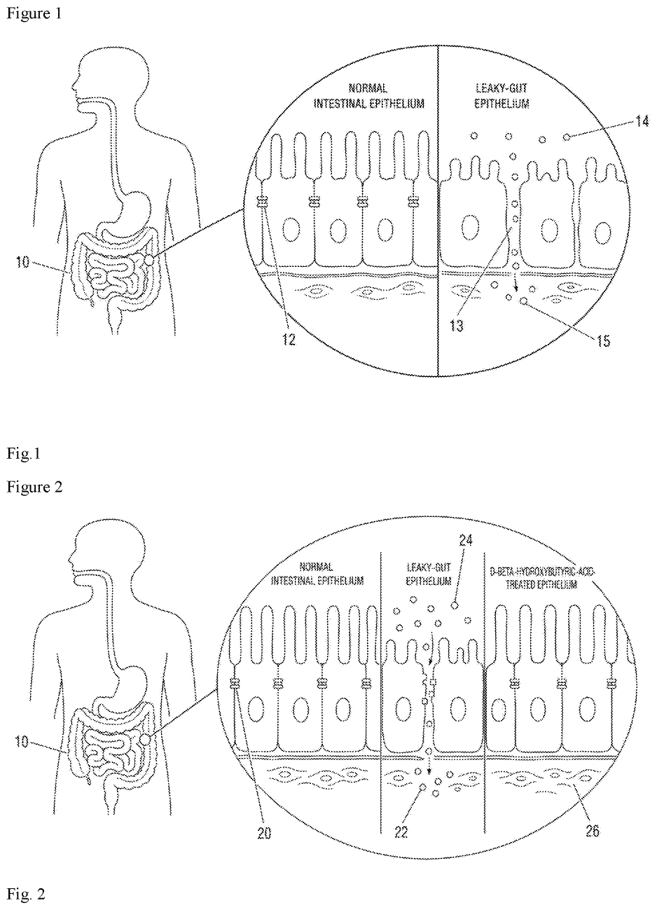

FIG. 2 schematically illustrates restoration of intestinal tight junctions by D-β-hydroxybutyric acid. Exogenous β-hydroxybutyrate has been reported in dextran sodium sulfate (DSS) colitis models to reduce inflammatory signaling (for example via NFκB and NLRP3), to normalize tight junction proteins such as ZO-1, occludin, and claudins, and to improve epithelial barrier structure and function. In contrast to previously known exogenous ketone formulations such as D/L ketone salts, ketone esters, and 1,3-butanediol, direct administration of bioidentical, salt-free D-β-hydroxybutyric acid provides rapid systemic availability without requiring gastric or hepatic conversion to release the active ketone body and may therefore offer superior effectiveness and tolerability.

A human torso is shown with the intestinal tract and an enlarged detailed view of the intestinal epithelium. Within the enlarged detail view, a row of intestinal epithelial cells bearing villi is depicted. A left region represents a healthy gut with normal villi 20 and tight junctions in a closed state. A central region depicts a leaky gut condition in which damaged junctions 22 are opened and lumen particles 24 pass between adjacent epithelial cells into underlying tissue. A right region depicts a D-β-hydroxybutyric-acid-treated gut in which junctions are restored and improved gut integrity 26 is achieved, with epithelial cells in close apposition and taller, more uniform villi, representing re-established barrier integrity.

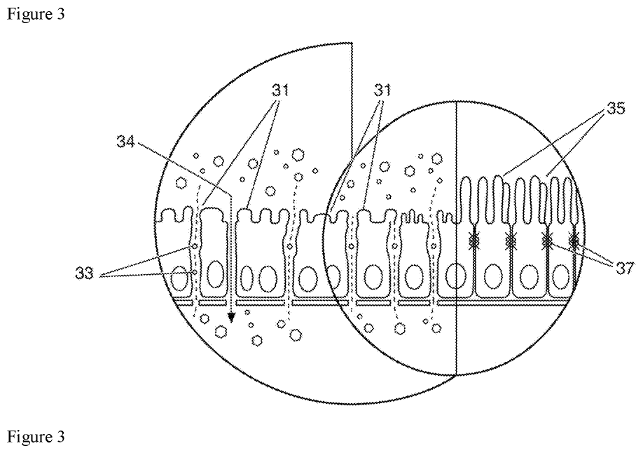

FIG. 3 schematically illustrates improvement of intestinal tight junction integrity and reduction of paracellular leakiness in response to administration of D-beta-hydroxybutyric acid in combination with vitamin B6. An enlarged view of the intestinal epithelium shows, on the left, a leaky-gut state associated with vitamin B6 deficiency characterized by blunted villi 31, disrupted junctions 33 between adjacent epithelial cells, and luminal particles 34 passing between cells into underlying tissue. In the middle region, incremental improvement is shown with administration of vitamin B6 alone to offset gut impact of vitamin B6 deficiency. On the right, the same epithelium is shown after treatment with D-beta-hydroxybutyric acid in combination with vitamin B6, with taller, more uniform villi 35, restored junctions 37, and substantially impeded passage of luminal particles representing improved barrier integrity that may result from synergistic or complementary effects of D-beta-hydroxybutyric acid and vitamin B6 on intestinal junction structure and function.

It is worth noting a distinguishing enhancement offered by utilizing bioidentical D-beta-hydroxybutyric acid in combination with vitamin B6. Bioidentical D-beta-hydroxybutyric acid is immediately bioavailable, providing substantially immediate positive impact on gut health with high bioavailability. This is distinguishable and in direct contrast to alternative potential embodiments utilizing other ketone-related agents such as ketone salts, ketone esters, 1,3-butanediol alone or in combination with vitamin. These other agents may still be used in alternative, less-preferred embodiments without departing from the spirit or scope of the invention, albeit with more limited efficacy due at least in part to the fact that such alternative ketogenic compounds require stomach chemical dissociation (and, in the case of ketone esters and ketone alcohol-derived products, additional liver post-processing), and thus may not be fully dissociated in the intestinal lining, resulting in unwanted mineral load and unavoidable metabolic burden thereby more rapidly restoring tight junction integrity 37 compared with these alternative agents.

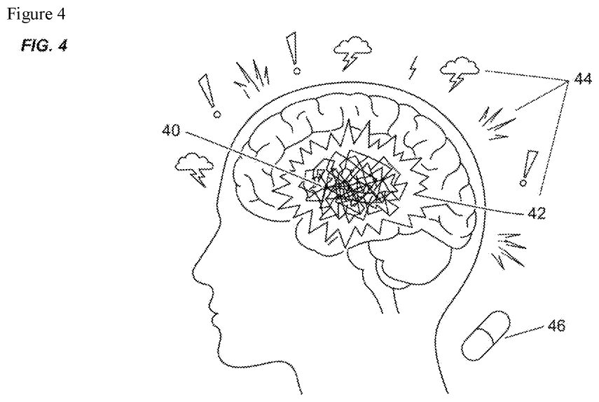

FIG. 4 depicts a graphical illustration of adverse neurophysiological effects associated with centrally acting pharmaceutical agents. A profile view of a subject's head shows a brain with a central region of disorganized, jagged line structures 40, representing disrupted and dysregulated neuronal network activity during exposure to, or withdrawal from, adverse neurophysiological effects associated with central nervous system (CNS)-active pharmaceuticals. Surrounding this region, a spiculated aura 42 visually represents heightened neuronal excitability and network instability. Peripherally, stress symbols 44, including exclamation marks, lightning bolts, and storm cloud icons, depict mood and behavioral manifestations such as irritability, anxiety, dysphoria, and agitation that are frequently reported as neuropsychiatric adverse events during treatment with antiepileptic and other central nervous system (CNS)-active agents. A pill or capsule element 46 represents such CNS-active pharmaceuticals, including, by way of non-limiting example, levetiracetam, selective serotonin reuptake inhibitors (SSRIs), and benzodiazepine derivatives, which can contribute to the network disruption 40 and instability 42 as illustrated. In preferred embodiments of the invention described herein, compositions comprising D-beta-hydroxybutyrate and pyridoxal-5-phosphate are administered as adjunctive nutritional therapy with the objective of stabilizing neuronal network activity, supporting GABAergic tone, and mitigating these neuropsychiatric adverse effects during both ongoing treatment and structured deprescription of such agents. The therapeutic effects of the claimed compositions are combinatorial and synergistic, leveraging enhanced intestinal barrier stability together with improved central nervous system network resilience.

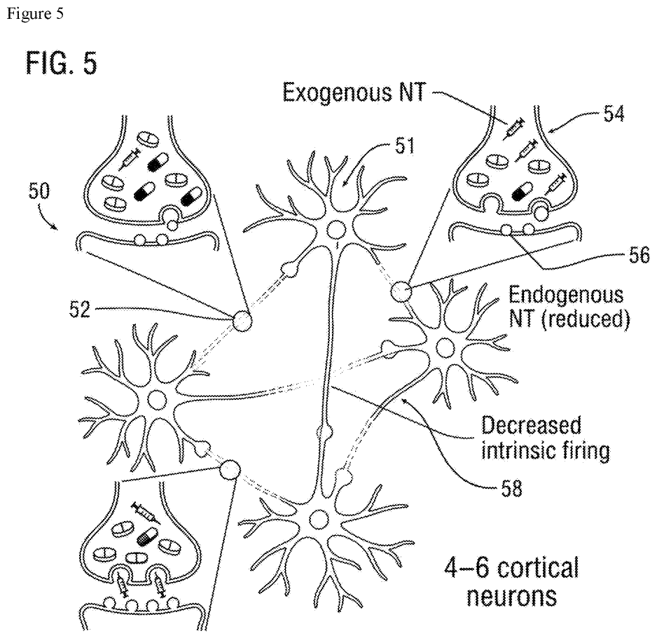

FIG. 5 is a schematic illustration 50 of a cortical neural network under chronic exposure to pharmaceutically administered, exogenous neurotransmitters, showing reduced endogenous neurotransmitter signaling and decreased intrinsic neuronal firing. A small network of cortical neurons 51 is interconnected by synapses 52. Peripheral presynaptic terminals 54 represent synapses in which neurotransmitter tone is dominated by exogenous neurotransmitter delivered by a pharmaceutical agent, as indicated by stylized pill and vial icons within the terminals. Within the central network, endogenous neurotransmitter release 56 from the cortical neurons 51 is reduced relative to the exogenous input 54, consistent with a state of pharmacologically altered neurotransmission in which intrinsic signaling is down-regulated. As a result, intrinsic neuronal firing activity 58 within the network is decreased, which is depicted by fewer and thinner dashed lines representing action potentials propagating along neuronal processes and by reduced synaptic signaling between neurons 51 at the synapses 52. The figure illustrates a brain state in which sustained administration of exogenous neurotransmitter-modulating drugs shifts the balance of neurotransmission toward exogenous input 54, with concomitant reduction of endogenous neurotransmitter output 56 and decreased intrinsic firing 58, thereby increasing network-level vulnerability during dose modification or withdrawal of such CNS-active agents.

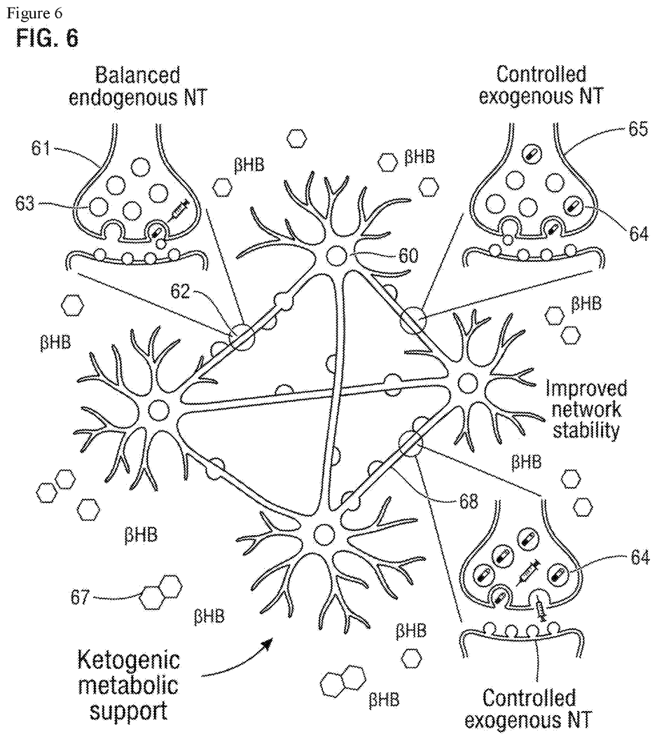

FIG. 6 is a schematic illustration of a cortical neural network receiving ketogenic metabolic support from D-β-hydroxybutyric acid (D-BHB) and exhibiting improved stability of neurotransmission. In the illustrated embodiment, a group of interconnected cortical neurons 60 forms a local network with synapses 62 between neuronal processes. Surrounding synaptic terminals 61 represent sites of predominantly endogenous neurotransmitter release, where vesicles containing neurotransmitter 63 are balanced and intrinsic firing within the network maintains physiological signaling tone. Other terminals 65 represent controlled exogenous neurotransmitter input, for example from centrally acting pharmaceutical agents, in which exogenous neurotransmitter 64 is present but regulated so as not to overwhelm endogenous signaling. Molecules of D-BHB 67 are depicted as hexagonal icons distributed around and within the network, indicating provision of ketogenic metabolic support to the neurons 60. In response to this metabolic support, endogenous neurotransmission remains balanced, exogenous neurotransmitter input 64 is maintained in a controlled range, and intrinsic network firing 68 (represented by continuous lines and closed synaptic loops) is stabilized, consistent with reports that ketone bodies and ketogenic interventions can enhance brain network stability and support more robust neural communication under metabolic or pharmacologic stress.

In certain embodiments, co-administration of vitamin B6 with D-β-hydroxybutyric acid may provide complementary or synergistic benefits by supporting pyridoxal-5′-phosphate-dependent synthesis of key neurotransmitters (including GABA, serotonin, and catecholamines), attenuating neuroinflammation and oxidative stress, and thereby further stabilizing excitatory-inhibitory balance and network-level brain activity beyond that achieved with D-β-hydroxybutyric acid alone.

In one embodiment, D-β-hydroxybutyric acid (D-BHB) is co-administered with a centrally acting pharmaceutical, either alone or in combination with vitamin B6, to provide alternative ketone-based fuel and reduced neuro-inflammation to the brain, stabilize large-scale neural network dynamics, and thereby reduce unwanted rapid mood swings, negative ideation, and other behavioral disturbances associated with the pharmaceutical's use. In related embodiments, D-BHB (optionally together with vitamin B6) is introduced as an active “deprescription ramp” during gradual dose reduction of the pharmaceutical, such that sustained ketone availability and metabolic support buffer the brain against energetic stress and network destabilization that can accompany withdrawal, helping to maintain excitatory-inhibitory balance and lowering the risk of rebound symptoms or other neurologic injury. Vitamin B6, by supporting pyridoxal-5′-phosphate-dependent synthesis of neurotransmitters including GABA, serotonin, and catecholamines, may further complement the neurometabolic actions of D-BHB, contributing to more orderly neurotransmission and smoother mood and cognition during both ongoing treatment and tapering.

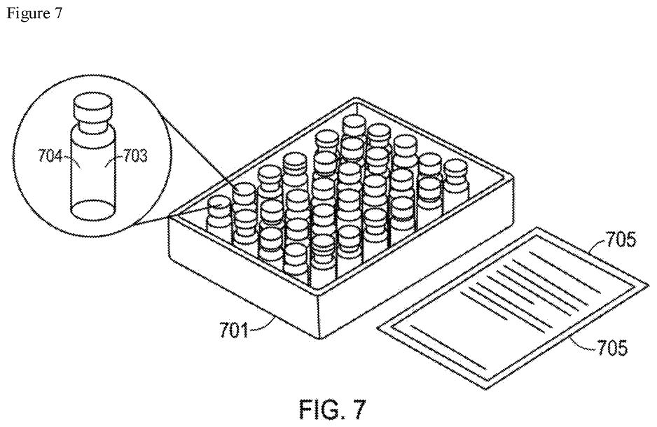

FIG. 7 illustrates an embodiment of an administration kit (701) for inducing exogenous ketosis in a human subject during ongoing treatment with, or structured tapering or recent discontinuation of, psychotropic agents such as antiepileptic drugs, selective serotonin reuptake inhibitors (SSRIs), and benzodiazepines. The kit comprises a plurality of unit-dose containers (703) of D-beta-hydroxybutyric acid, each containing a salt-free, alcohol-free, ester-free bioidentical D-beta-hydroxybutyric acid composition in an amount suitable for a single oral administration. In preferred embodiments, each unit dose contains between about 5 g and about 20 g, more preferably between about 5 g and about 10 g, of net bioidentical D-beta-hydroxybutyric acid. The container (703) may additionally and optionally contain a therapeutically effective amount of a B vitamin (704), wherein vitamin B6 is a preferred embodiment, but alternative variants such as vitamin B12 may also be employed. The kit further comprises printed or electronic instructions for use (705) directed to an end user or clinician, specifying that unit doses are to be administered to raise blood D-beta-hydroxybutyrate to a ketotic range of at least about 0.3 mmol/L. In certain embodiments, the instructions for use (705) are provided at least in part via a machine-readable element such as a QR code or similar linking object. In a preferred embodiment, D-beta-hydroxybutyric acid is administered in the range of about 0.25 g/kg/day to about 1.0 g/kg/day, with guidance for adjustment based on clinical response and, in some embodiments, AI-assisted response monitoring as described herein.

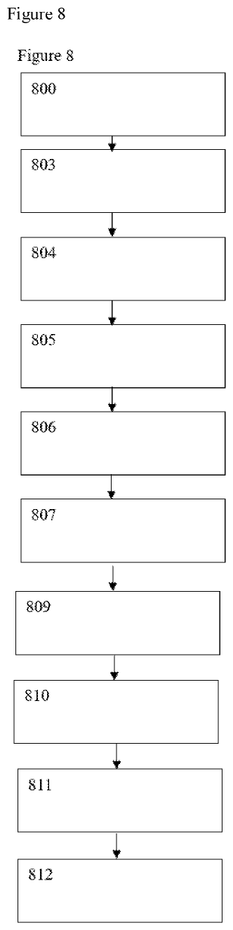

FIG. 8 details a variety of method steps by which the claimed invention may be practiced. In its simplest form, an adult subject in need of neurological stabilization assesses an appropriate dose (800) and consumes 812 a 5 g to 30 g drink of bioidentical D-beta-hydroxybutyric acid, optionally with one or more B vitamins, diluted in approximately 300 mL to 500 mL of water for palatability. In certain embodiments, the drink is prepared from one or more unit-dose containers of D-beta-hydroxybutyric acid, optionally including a B vitamin component such as vitamin B6. The physiological benefits of D-beta-hydroxybutyric acid intake include improved intestinal barrier integrity, rapid signaling along the vagus nerve to the brain, efficient metabolism across the blood-brain barrier with enhanced bioenergetics and increased neural network resilience. These benefits support subjects who are maintaining, tapering, or have recently discontinued psychotropic agents, and are complementary to, and not limited by, the specific mechanisms summarized above. A key attribute of the bioidentical D-beta-hydroxybutyric acid formulations described herein is their immediate bioavailability in the stomach, gastrointestinal tract, and brain, distinguishing them from ketone salts and ketone ester or 1,3-butanediol products that impose mineral load and hepatic processing requirements and may include racemic D+L mixtures. Improved palatability of bioidentical D-beta-hydroxybutyric acid relative to ketone salts and ketone alcohol formulations further supports voluntary adherence.

In a preferred embodiment, the pharmaceutical target for de-prescription (at initial prescription size or incremental reduced ‘de-prescribe’ amount) is provided in pill or capsule form together with powdered D-Beta Hydroxybutyric Acid and optional vitamin B6 in a single capsule. While this may alternatively be achieved in liquid format, providing a comprehensive ‘all in one’ pill is preferred to promote adherence and reduce potential misadministration.

Strategies to raise ketones such as a ketogenic diet along with bioidentical D-BHB acid result in improved seizure threshold both for traditional epileptics as well as pharmaceutical reliant seizure withdrawal. While the underlying mechanisms remain unclear (and as yet not thoroughly independently validated) it is likely that improved brain network stability either alone or together with re-establishment of gut barrier integrity results in improved seizure threshold levels. With gut repair and better gut barrier mucosal integrity it is likely that GABA synthesis raises the GABA/glutamate ratio which consequently reduces seizure susceptibility through improved gut-brain axis communication and resiliency. Foreseeable improvements in the claimed invention include precision guidance of nutritional factors which can have an impact due to gut dysbiosis and inflammation.

The bioidentical D-BHB acid alone or in combination with B1, B6, B9, B12 can work to raise the ketones and reestablish the gut barrier integrity. With gut repair and better barrier mucosal integrity there is reduced dysbiosis, better vagal tone and less inflammation. Improving gut resiliency is important since in a healthy human the gut makes 90% of serotonin, 70% of dopamine and 30% of GABA. B6 in preferred embodiment of the claimed invention utilizes pyridoxal 5 phosphate (known as P5P or PYP hereinafter) is optimal but alternative embodiments may utilize pyridoxine which is more common but may result in neuronal damage if not managed properly. In a preferred embodiment Vitamin B6 is utilized to improve the seizure threshold. As a potential mechanism it may help to maintain healthy gut bacteria and reduce gut dysbiosis. B6 is also a cofactor in the healthy normal production of serotonin, dopamine and GABA.

For anti-epileptic pharmaceuticals, healthy gut function leading to normal serotonin, dopamine and GABA is important for management of levetiracetam (among other pharmaceuticals) side effects. Vitamin B6 deficiency has been known to excite seizures and irritability. According to the claimed invention, combination of bioidentical D-BHB together with B6 supplementation is expected to lessen levetiracetam irritability and instability in both vitamin B6 deficient as well as vitamin B6 normal subjects. Anxiolytics and antidepressants like but not limited to SSRIs (selective serotonin reuptake inhibitor) increase synaptic serotonin, yet over time SSRI pharmaceuticals deplete B6 consequently diminishing a key cofactor for the healthy function of the gut brain axis leading to side effects like irritability. Supplementing B6 according to the claimed invention is expected to support smoother serotonin regulation (and consequent pharmaceutical de-prescription) without directly amplifying SSRI negative effects.

Bioidentical D-BHB is both direct mitochondrial energy as well as a known anti-inflammatory. It can fuel the gut-brain axis, while folate, betaine, and B12 help renew the gut lining and keep methylation working smoothly and support a healthier gut brain axis. The claimed combination alleviates nervous system stress from anti-epilepsy compounds, antidepressants and anxiolytics. It is an important aspect of the claimed invention that the physiological effects are nutritional in nature, not pharmacological. Consequently, as the ameliorative agents are fully and naturally consumed there is no replacement of one pharmacological dependency with another.

Between the initial dose-assessment step (800) and the consumption step (812) a range of optional therapeutic interventions may be implemented. An optional dietary data capture step (803) uses smartphone or comparable imaging to record food intake, for example by photographing meals with a body-scaled reference such as the user's hand to aid portion estimation. An optional pharmaceutical data capture step (804) uses similar imaging to record prescribed medications, enabling missed-dose warnings and basic assessment of pharmaceutical dose reductions over time. An optional real time data acquisition step (805) captures information from blood ketone strips, glucose test strips, acetone breath analyzers, or continuous ketone monitors. The real time data acquisition step may additionally be combined with an optional remote lab testing data acquisition step (806) captures more comprehensive lab data. Including but not limited to data from steps (803), (805) and (806) may be processed in an optional diet calculation step (807) in which an AI system provides individualized dietary optimization guidance that improves over time with continued subject feedback and anonymized aggregate data from other users, including accommodation of dietary constraints such as celiac disease. Data from steps (804) and (805) may be processed in an optional pharmaceutical dosage calculation step (808), in which an AI system assists in optimizing psychotropic dosing and tapering trajectories. In preferred embodiments, metabolic parameters, diet guidance, and dosage interpretation are sex-specific; for example, implementations for women may incorporate menstrual cycle data (809) rather than treating women as scaled-down males. Because the psychotropic agents of interest directly influence mood, an optional mood assessment step (811) employs active measures such as validated mood scales (e.g., Beck-type instruments) and, in some embodiments, passive measures such as analysis of voice characteristics to detect blunted affect or emerging depressive features that may not be recognized by the subject. Optional missed pharmaceutical alerts and diet-enhancement prompts (810) can be generated before consumption step (812) or at any point during use of the system.

Over time, the method aims to provide optimal mood support while the subject is receiving psychotropic medication, followed by the safest feasible taper and post-taper monitoring. Because the period following discontinuation of psychotropic agents is often clinically vulnerable, it is a foreseeable embodiment that the full system, including D-beta-hydroxybutyric acid administration and associated data-driven guidance, is continued for several months or longer after pharmaceutical cessation.

### Example 1 (Prophetic): Co-Administration of Bioidentical D-β-Hydroxybutyric Acid with Vitamin B6 During SSRI Deprescription

1.1 Subject. A 52-year-old female subject of 70 kg body weight, having received a selective serotonin reuptake inhibitor (SSRI) at a stable maintenance dose for at least 12 months, is enrolled in a supervised deprescription protocol. Data capture includes capillary blood BHB ketone and glucose based on a fasted state on waking in morning for greater than 25 days during 30 days prior to taper, capillary blood β-hydroxybutyrate (BHB) greater than or equal to 0.6 mmol/L, capillary blood glucose below 100 mg/dL. Ideal target is a Weekly Beck Depression Inventory-II (BDI-II) score within a clinically stable range. The subject has no contraindication to ketogenic intervention and no history of type 1 diabetes or inborn error of fatty acid oxidation. Subject follows a low carbohydrate diet of less than 100 g of carbs per day, without snacks and not more than three meals per day for at least one month prior to taper, during taper and for no less than 3 months post taper. Subject is tested for B6, B9, B12 and homocysteine before taper.

1.2 Composition administered. Each unit dose comprises 5-10 g of bioidentical D-β-hydroxybutyric acid in salt-free, ester-free, and alcohol-free form, together with 5 -25 mg of vitamin B6 (preferentially pyridoxal 5 phosphate or alternative pyroxidine, vitamin B9 (using preferred methylfolate alternatively folic acid), vitamin B12 (prefer methyl cobalamin alternatively cobalamin) and betaine (TMG) provided as a liquid concentrate suitable for dilution. Each unit dose is diluted in 300-800 mL of cold water immediately prior to consumption to improve palatability and support voluntary adherence. Night time unit is half the dose of daytime dose if this is greater than 5 g of bioidentical D-BHB alternatively it is 5 g of bioidentical D-BHB and is without B vitamins or betaine.

1.3 Dosing regimen. The subject is administered the unit dose orally three times per day, corresponding to a total daily dose of 15 g to 30 g of D-β-hydroxybutyric acid (approximately 0.43 g/kg/day for a 70 kg subject), within the disclosed range of 0.25 g/kg/day to 1.0 g/kg/day. Doses are taken approximately 60-90 minutes before breakfast, before lunch, and 2 hours before bedtime. As alternate preferred embodiment, D-BHB is additionally administered prior to 60-90 minutes of falling asleep, the additional sleep dosage aids deprescription by increasing deep sleep and optimizing the glymphatic system natural brain waste filtration effects. Ideal administration includes no food snacks between meals and no more than 3 meals per day.

1.4 Pharmacokinetic targets. Following each oral dose, capillary blood BHB is expected to rise above the ketotic threshold of 0.6 mmol/L within approximately 10-20 minutes, to reach a peak concentration of approximately 1.0-2.5 mmol/L between 30 and 60 minutes post-dose, and to remain at or above 0.6 mmol/L for at least 2 hours post-dose. With three-times-daily administration, time-averaged blood BHB is expected to remain above 0.6 mmol/L for at least 12 hours per 24-hour period during the active treatment phase. As alternate preferred embodiment, D-BHB is additionally administered prior to 90 minutes of falling asleep. The additional sleep dosage aids deprescription by increasing deep sleep and optimizing the glymphatic system natural brain waste filtration effects.

1.5 Stabilization phase (Days 1-30). D-β-hydroxybutyric acid and/or vitamin B6, B9, B12 and Betaine are administered as described in § 1.3 while the SSRI is maintained at the prior maintenance dose. Capillary blood BHB is measured by test strip at least once daily between 4-6 pm at least three hours post meal, and the measurement is captured by smartphone camera and transmitted to the guidance system described herein. Capillary blood glucose is measured at least once daily by analogous means. Mood is assessed daily using the BDI-II administered through the smartphone interface, and passive voice-tone monitoring is enabled to detect deviations from the subject's stable-mood baseline and passive touch type monitoring, may be enabled to detect subject's mood stability.

1.6 Deprescription ramp (Days 31-180) . Beginning on Day 31, the SSRI dose is reduced in accordance with the prescribing clinician's taper schedule, by way of non-limiting example a 2-25% reduction every 14 days, while D-β-hydroxybutyric acid administration is continued at 15-30g/day as in § 1.3. The taper will be parabolic with the dose reduction higher in initial taper and a long tail. The dosage-guidance system described herein adjusts D-β-hydroxybutyric acid timing and, where indicated, individual unit-dose magnitude in response to (a) measured blood BHB trending below 0.6 mmol/L during waking hours, (b) BDI-II score increases of 6 points or more above baseline, or (c) voice-feature deviation flags indicative of flattened affect, passive touch monitoring flags instability. In a female subject as in this Example, the guidance system applies sex-specific parameters that take menstrual-cycle phase as an input, increasing inter-dose frequency during the late luteal phase to maintain blood BHB above 0.6 mmol/L through periods of greater mood lability.

1.7 Post-cessation continuation phase (Days 85-265). Following complete cessation of the SSRI on or about Day 85, administration of D-β-hydroxybutyric acid with vitamin B6 is continued at 0.25-0.5 g/kg/day for at least an additional 90 days, and in some embodiments at least 180 days, to support neural network stability through the post-deprescription window during which rebound symptoms are most likely to occur. As D-BHB may also be tapered, it is likely important to maintain the 5 g dose 90 minutes before bedtime for at least three months to help increase brain network stability and proper function of the glymphatic system with enhanced deep sleep.

1.8 Expected outcomes. Deprescribing SSRI use in the absence of the claimed invention (either by immediate cessation or unguided taper) is not just suboptimal, it can be substantially dangerous to the health of the individual. Doing so can lead to more than 20% of individuals developing SSRI withdrawal syndrome commonly described by the mnemonic FINISH.

FINISH is a cluster of symptoms including Flu like symptoms, Insomnia, Nausea (variety of digestive issues), Imbalance (dizziness and gait problems), Sensory issues (‘brain zaps’ rolling waves of pain and seeing colors) Hyperarousal (anxiety, aggression and agitation). These symptoms can be permanent and frequently presents as a relapse of depression, and the patient is put back on the SSRI. Use of bioidentical D-BHB for SSRI deprescription tapering has already led to unexpected benefits including overcoming headache and dizziness. Moreover, cessation of SSRI medication went in specific user experiences from “doubtful, difficult or impossible” to “possible.”

With bioidentical D-BHB according to the claimed invention SSRI discontinuation syndrome is avoided by many individuals wishing to discontinue SSRI therapy for depression, PTSD and anxiety among other conditions. With Bioidentical D-BHB and optional B vitamin supplementation, the cluster of symptoms known as FINISH including Flu like symptoms, Insomnia, Nausea (variety of digestive issues), Imbalance (dizziness and gait problems), Sensory issues (‘brain zaps’ rolling waves of pain and seeing colors) Hyperarousal (anxiety, aggression and agitation) are blunted and mitigated to the point where long term cessation of SSRI without harm by controlled taper is now possible.

Moreover, in a further unexpected benefit, successful SSRI deprescription according to the claimed method is anticipated to mitigate SSRI-associated sexual dysfunction in a substantial proportion of individuals, including reduced libido, anorgasmia, difficulty with arousal, erectile difficulties, and genital hypoesthesia that commonly emerge during long-term serotonergic antidepressant treatment. By maintaining mood stability, minimizing withdrawal-related hyperarousal, and allowing gradual reduction of serotonergic interference with dopaminergic and noradrenergic pathways involved in sexual response, the disclosed D-β-hydroxybutyrate-supported taper is expected to permit partial or complete recovery of sexual function in many subjects who would otherwise remain symptomatic or be maintained indefinitely on SSRIs despite distressing sexual side effects. Although rare cases of persistent post-SSRI sexual dysfunction have been described and may not fully resolve despite drug cessation, structured deprescription with metabolic and micronutrient support may reduce the incidence and severity of enduring symptoms relative to unguided discontinuation, thereby improving quality of life and facilitating long-term SSRI-free remission.

It is expected that, relative to a comparable deprescription protocol conducted without D-β-hydroxybutyric acid administration
(a) the subject maintains capillary blood BHB ≥0.6 mmol/L for a time-averaged majority of waking hours throughout the deprescription ramp;
(b) BDI-II scores remain within 6 points of the subject's pre-taper baseline throughout the deprescription ramp and the post-cessation continuation phase;
(c) no clinically significant withdrawal-associated adverse events occur during the deprescription ramp;
(d) capillary blood glucose remains within the subject's pre-treatment range preferable under 100 upon waking, indicating that the metabolic support is provided by exogenous ketone availability rather than by induced hypoglycemia; and
(e) gastrointestinal tolerability is improved relative to administration of an equimolar dose of a D/L ketone salt, an equimolar dose of a ketone monoester, or an equimolar dose of 1,3-butanediol, by reason of the absence of counter-ion load, ester hydrolysis byproducts, and hepatic alcohol-to-ketone conversion intermediates, respectively.

1.9 Non-limiting nature. The dose magnitudes, dose frequencies, blood BHB targets, time-to-onset windows, taper schedule, and continuation duration set forth in this Example are illustrative and non-limiting. In other embodiments, unit doses range from 2 g to 40 g, daily totals range from 0.1 g/kg/day to 1.5 g/kg/day, blood BHB targets range from 0.3 mmol/L to 5.0 mmol/L, and post-cessation continuation extends from 30 days to 12 months or longer, without departing from the scope of the invention.

Example 2 (Prophetic): Co-administration of Bioidentical D-β-Hydroxybutyric Acid with Vitamin B6 During Levetiracetam (Keppra) Maintenance and Taper in a Young Adult Female Subject

2.1 Subject. A 19-year-old female subject of 50 kg body weight, having received levetiracetam (Keppra) at a stable maintenance dose of 250 mg orally twice daily (500 mg total daily dose) for at least 6 months for control of a focal-onset seizure disorder, is enrolled in a supervised neurometabolic support protocol. Baseline assessments include capillary blood β-hydroxybutyrate (BHB) of less than 0.2 mmol/L, capillary blood glucose of 80-95 mg/dL, a Beck Depression Inventory-II (BDI-II) score within a clinically stable range, a documented seizure diary spanning at least the prior 90 days, and characterization of menstrual-cycle regularity and phase. The subject has no contraindication to ketogenic intervention, no history of type 1 diabetes, no inborn error of fatty acid oxidation, and is not pregnant. While 250 mg orally twice daily (500 mg total daily dose) is used in this illustrative example, the disclosed taper rate may be similarly applied to other individuals taking 500 mg or 1000 mg levetiracetam twice daily.

2.2 Composition administered. Each unit dose comprises 7.5 g of bioidentical D-β-hydroxybutyric acid in salt-free, ester-free, and alcohol-free form, together with 5 mg of preferred pyridoxal-5-phosphate or alternatively pyridoxine hydrochloride (vitamin B6), provided as a liquid concentrate suitable for dilution. Each unit dose is diluted in 300-800 mL of cold water immediately prior to consumption to improve palatability and support voluntary adherence. In the present Example, the smaller 7.5 g unit dose, relative to the 10 g unit dose of Example 1, is selected to accommodate the lower body mass of the subject and to permit finer-grained alignment of BHB peaks with peri-dose levetiracetam troughs as described below.

2.3 Dosing regimen. The subject is administered the unit dose orally four times per day, corresponding to a total daily dose of 30 g of D-β-hydroxybutyric acid (0.6 g/kg/day for a 50 kg subject), within the disclosed range of 0.25 g/kg/day to 1.0 g/kg/day. Doses are timed to bracket the pharmacokinetic trough windows of twice daily levetiracetam administration and to maintain ketotic support across the overnight period during which seizure susceptibility is, in many subjects, elevated. By way of non-limiting example, the subject's levetiracetam doses are taken at approximately 07:00 and 19:00, and D-β-hydroxybutyric acid unit doses are administered at approximately 06:30 (pre-AM-Keppra), 12:30 (mid-day), 18:30 (pre-PM-Keppra), and 23:00 (pre-sleep).

2.4 Pharmacokinetic targets and intra-day seizure-threshold-calibrated profile. Following each oral dose, capillary blood BHB is expected to rise above the ketotic threshold of 0.6 mmol/L within approximately 10-20 minutes, to reach a peak concentration of approximately 0.8-2.0 mmol/L between 30 and 60 minutes post-dose, and to remain at or above 0.6 mmol/L for at least 2 hours post-dose. The four-times-daily regimen of § 2.3 is configured such that:
(a) blood BHB is at or above 0.6 mmol/L during each of the two peri-dose levetiracetam trough windows (approximately the 60-minute interval immediately preceding each scheduled levetiracetam dose), during which serum levetiracetam is at its lowest concentration and seizure-threshold reserve is correspondingly reduced;
(b) blood BHB is at or above 0.6 mmol/L for at least the first 4 hours of the overnight period following the 23:00 dose, providing metabolic support during the early-sleep interval associated in many subjects with elevated seizure susceptibility;
(c) time average blood BHB across the 24-hour period remains at or above 0.6 mmol/L for at least 8 hours per day during the active treatment phase; and
(d) no individual unit dose is configured to drive peak blood BHB above 5.0 mmol/L, to avoid unnecessary excursion beyond the therapeutic window and to preserve gastrointestinal tolerability and palatability-driven adherence.

2.5 Stabilization phase (Days 1-28). D-β-hydroxybutyric acid and vitamin B6 are administered as described in § 2.3 while levetiracetam is maintained at initial dose twice daily. Capillary blood BHB is measured by test strip at least twice daily—once during a peri-dose levetiracetam trough window and once at a non-trough time—and each measurement is captured by smartphone camera and transmitted to the dosage-guidance system described herein. In additional embodiments, continuous ketone monitoring by an interstitial-fluid patch sensor is substituted for, or supplemented with, the capillary strip measurements, providing the guidance system with a substantially continuous BHB time series. Capillary blood glucose is measured at least once daily by analogous means. Mood is assessed daily using the BDI-II administered through the smartphone interface, and passive voice-tone monitoring is enabled to detect deviations from the subject's stable-mood baseline and touch typing is monitored for emotional stability. Seizure events, auras, and any breakthrough symptoms are logged contemporaneously through the smartphone interface, and menstrual-cycle phase is tracked. Carbohydrate consumption remains below 50 g per day with no snacks between meals with either 2 or 3 meals per day.

2.6 Sex- and cycle-specific dosage adjustment. The dosage-guidance system described herein applies sex-specific parameters that take menstrual-cycle phase as an input. In the present Example, in which the subject is a young adult female, the guidance system identifies the peri-menstrual window (defined for purposes of this Example as the interval beginning approximately 3 days prior to onset of menses and extending through the second day of menses) as a phase of elevated catamenial seizure susceptibility, and during this window the system increases the frequency or magnitude of D-β-hydroxybutyric acid administration so as to (i) raise the time-averaged blood BHB floor by at least 0.1 mmol/L relative to the mid-follicular baseline, and (ii) extend the duration during which blood BHB remains at or above 0.6 mmol/L by at least an additional 2 hours per 24-hour period. Such adjustment is implemented, by way of non-limiting example, by adding a fifth unit dose of 5 -7.5 g at approximately 03:00 during the peri-menstrual window, or by increasing the 23:00 unit dose from 7.5 g to 10 g during that window, or both. The same algorithmic structure is applied with sex-specific parameters distinct from those used in male subjects, consistent with the disclosure that calculations involving female subjects incorporate menstrual-cycle timing rather than treating female subjects as scaled male subjects. Bioidentical D-BHB dosage should be evaluated and reduced once new habits of no snacking, nutrient dense meals, walking and sleep are implemented to facilitate the endogenous production of ketones along with exogenous supplementation. After a period of endogenous ketosis is achieved a more natural schedule of midmorning, mid afternoon and 2 hours before bed can be considered prior to the optional deprescription ramp. While 28 days is proposed in this example, a longer lead in for consideration of deprescription may be preferable.

2.7 Deprescription ramp (Days 29-168). Beginning on Day 29, and only upon clinical determination by the prescribing neurologist that taper of levetiracetam is appropriate, levetiracetam is reduced in accordance with the clinician's taper schedule. By way of non-limiting example, levetiracetam total daily dose is reduced by approximately 10% of the then-current dose every 14 to 28 days, with the inter-step interval lengthening as the dose decreases (e.g., 1.0 →0.9→0.8→0.7→0.6→0.5→0.4→0.3→0.25→0.2→0.15→0.125→0.1→0.0625→0.03125→0 mg/day), with intermediate dose magnitudes achieved by liquid titration or by compounded micro-tablets. D-β-hydroxybutyric acid administration is continued at 30 g/day as in § 2.3 and is adjusted intra-day in accordance with § 2.4 and § 2.6. The dosage-guidance system increases the time-averaged blood BHB floor during taper by, for example, advancing the 06:30 and 18:30 unit doses by an additional 15-30 minutes ahead of the corresponding (reduced) levetiracetam dose, and by maintaining the § 2.6 peri-menstrual augmentation throughout the taper. Should the seizure log register a breakthrough event, an aura, or a clinically significant EEG change, the guidance system flags the event for the prescribing clinician and recommends pausing further levetiracetam reduction and increasing D-β-hydroxybutyric acid time-averaged exposure pending clinical review. A longer nonlinear deprescription ramp should also be considered in the context of any past withdrawal seizure experiences of the patient along with the capacity to self regulate lifestyle factors of food, exercise and sleep.

2.8 Post-cessation continuation phase (days 169 Et Seq.). Following complete cessation of levetiracetam, in those subjects for whom cessation is clinically appropriate, administration of D-β-hydroxybutyric acid with vitamin B6 is continued at 0.25-0.5 g/kg/day for at least an additional 90 days, and in some embodiments at least 180 days, to support neural network stability through the post-deprescription window during which breakthrough seizure activity, if any, is most likely to emerge. In subjects for whom levetiracetam is not tapered and is instead continued indefinitely as maintenance therapy, administration of D-β-hydroxybutyric acid with vitamin B6 in accordance with § 2.3 through § 2.6 is continued indefinitely as a neurometabolic adjuvant.

2.9 Expected outcomes. It is expected that without bioidentical D-BHB, attempting to taper or rapidly cease levetiracetam places the individual at substantially greater risk of withdrawal seizure. By utilizing the claimed invention the subject is expected to be able to cease levetiracetam at a gradual rate with greatly diminished to no side effects or withdrawal seizures. It is expected that, relative to a comparable maintenance or taper protocol conducted without D-β-hydroxybutyric acid administration a taper protocol with D-β-hydroxybutyric acid administration and vitamin b6:
(a) the subject maintains capillary blood BHB≥0.6 mmol/L during each peri-dose levetiracetam trough window and during at least the first 4 hours of the overnight period;
(b) the seizure-event rate during the maintenance phase is no greater than the subject's pre-treatment 90-day baseline rate, and the rate during the deprescription ramp is no greater than 1.5 times that baseline;
(c) catamenial clustering of seizure events, if present at baseline, is reduced in frequency or magnitude during peri-menstrual windows in which the § 2.6 cycle-specific augmentation is applied;
(d) BDI-II scores remain within 5 points of the subject's pre-treatment baseline, and voice-feature monitoring registers no sustained flattened-affect deviation lasting more than 72 hours;
(e) capillary blood glucose remains within the subject's pre-treatment range, indicating that the metabolic support is provided by exogenous ketone availability rather than by induced hypoglycemia; and
(f) gastrointestinal tolerability and palatability-driven adherence are improved relative to administration of an equimolar dose of a D/L ketone salt, an equimolar dose of a ketone monoester, or an equimolar dose of 1,3-butanediol, by reason of the absence of counter-ion load, ester hydrolysis byproducts, and hepatic alcohol-to-ketone conversion intermediates, respectively.

2.10 Non-limiting nature. The dose magnitudes, dose frequencies, intra-day timing relative to levetiracetam administration, blood BHB targets, peri-menstrual augmentation parameters, taper schedule, and post-cessation continuation duration set forth in this Example are illustrative and non-limiting. In other embodiments, unit doses range from 2 g to 40 g, daily totals range from 0.1 g/kg/day to 1.5 g/kg/day, dose frequencies range from twice daily to six times daily, blood BHB targets range from 0.3 mmol/L to 5.0 mmol/L, peri-menstrual BHB-floor augmentation ranges from 0.05 mmol/L to 0.5 mmol/L above baseline, and post-cessation continuation extends from 30 days to 12 months or longer, without departing from the scope of the invention. The methods of this Example are further applicable, with appropriate dose and timing adjustments, to other antiepileptic drugs including without limitation lamotrigine, valproate, carbamazepine, oxcarbazepine, topiramate, lacosamide, and zonisamide, and to subjects of either sex across the adult age range, with sex-specific parameters applied as described in § 2.6.

Example 3 (Prophetic): Co-administration of Bioidentical D-β-Hydroxybutyric Acid with Vitamin B6 During Long-Term Clonazepam Deprescription in a Middle-Aged Male Subject

3.1 Subject. A 58-year-old male subject of 85 kg body weight, having received clonazepam at a stable maintenance dose of 0.5 mg orally twice daily (1.0 mg total daily dose) for at least 8 years for management of generalized anxiety disorder with associated insomnia, is enrolled in a supervised deprescription protocol. Baseline assessments include capillary blood β-hydroxybutyrate (BHB) of less than 0.2 mmol/L, capillary blood glucose of 90-105 mg/dL, a Beck Depression Inventory-II (BDI-II) score within a clinically stable range, a State-Trait Anxiety Inventory (STAI) score within the subject's stable baseline, a documented sleep diary spanning at least the prior 30 days, and characterization of any prior taper attempts. The subject has no contraindication to ketogenic intervention, no history of type 1 diabetes, no inborn error of fatty acid oxidation, no active alcohol use disorder, and no concomitant opioid therapy.

3.2 Composition administered. Each unit dose comprises 12 g of bioidentical D-β-hydroxybutyric acid in salt-free, ester-free, and alcohol-free form, together with 25 mg of vitamin B6 (prefer pyridoxal 5 phosphate or alternative pyroxidine, vitamin B9 (prefer methylfolate alternatively folic acid), vitamin B12 (prefer methyl cobalamin alternatively cobalamin) and betaine (TMG), provided as a liquid concentrate suitable for dilution. Each unit dose is diluted in 300-800 mL of cold water immediately prior to consumption to improve palatability and support voluntary adherence. In the present Example, the 12 g unit dose, intermediate between the 10 g unit dose of Example 1 and the 7.5 g unit dose of Example 2, is selected to accommodate the higher body mass of the subject and to provide a sustained-coverage BHB exposure profile suitable for a centrally acting pharmaceutical of long elimination half-life as described below.

3.3 Dosing regimen. The subject is administered the unit dose orally three times per day, corresponding to a total daily dose of 36 g of D-β-hydroxybutyric acid (approximately 0.42 g/kg/day for an 85 kg subject), within the disclosed range of 0.25 g/kg/day to 1.0 g/kg/day. In contrast to the levetiracetam embodiment of Example 2, in which D-β-hydroxybutyric acid unit doses are timed to bracket the peri-dose pharmacokinetic trough windows of a twice-daily, short-trough antiepileptic, the present Example employs a sustained-coverage timing strategy appropriate for a benzodiazepine of long elimination half-life (clonazepam t½ approximately 30-40 hours), in which serum benzodiazepine concentration is comparatively flat across the inter-dose interval and the relevant vulnerability windows during taper are not narrow peri-dose troughs but rather sustained periods of reduced GABAergic tone. By way of non-limiting example, the subject's clonazepam doses are taken at approximately 08:00 and 20:00, and D-β-hydroxybutyric acid unit doses are administered at approximately 07:30 (morning or upon waking), 14:00 (mid-afternoon), and 22:00 (pre-sleep), so as to provide three temporally separated peaks of ketotic support across the waking and early-sleep periods.

3.4 Pharmacokinetic targets and sustained-coverage profile calibrated to gabaergic withdrawal vulnerability. Following each oral dose, capillary blood BHB is expected to rise above the ketotic threshold of 0.6 mmol/L within approximately 10-20 minutes, to reach a peak concentration of approximately 1.0-2.5 mmol/L between 30 and 60 minutes post-dose, and to remain at or above 0.6 mmol/L for at least 2 hours post-dose. The three-times-daily regimen of § 3.3 is configured such that:
(a) blood BHB is at or above 0.6 mmol/L during the morning awakening window (approximately the first 90 minutes after the subject's habitual wake time), during which serum benzodiazepine concentration has declined from its overnight steady state and during which rebound anxiety, autonomic activation, and cognitive dysphoria are most commonly reported by subjects undergoing benzodiazepine taper;
(b) blood BHB is at or above 0.6 mmol/L during the mid-afternoon interdose window (approximately 13:00-16:00), during which subjects on twice-daily benzodiazepine regimens commonly report inter-dose breakthrough anxiety;
(c) blood BHB is at or above 0.6 mmol/L for at least the first 4 hours of the overnight period following the 22:00 dose, providing metabolic support during the early-sleep interval and addressing taper-associated sleep-onset insomnia and early-cycle sleep fragmentation;
(d) time-averaged blood BHB across the 24-hour period remains at or above 0.6 mmol/L for at least 8 hours per day during the active treatment phase, with the time-averaged BHB floor increased during taper as set forth in § 3.7; and
(e) no individual unit dose is configured to drive peak blood BHB above 5.0 mmol/L, in order to avoid unnecessary excursion beyond the therapeutic window and to preserve gastrointestinal tolerability and palatability-driven adherence.
3.5 Stabilization phase (Days 1-28). D-β-hydroxybutyric acid and vitamin B6 are administered as described in § 3.3 while clonazepam is maintained at 0.5 mg twice daily. Capillary blood BHB is measured by test strip at least twice daily —once during the morning awakening window of § 3.4(a) and once during the mid-afternoon interdose window of § 3.4(b) —and each measurement is captured by smartphone camera and transmitted to the dosage-guidance system described herein. In additional embodiments, continuous ketone monitoring by an interstitial-fluid patch sensor is substituted for, or supplemented with, the capillary strip measurements, providing the guidance system with a substantially continuous BHB time series. Capillary blood glucose is measured at least once daily by analogous means. Mood and anxiety are assessed daily using the BDI-II and a brief STAI administered through the smartphone interface, and passive voice-tone monitoring is enabled to detect deviations from the subject's stable-affect baseline, with particular attention to elevations in vocal tension, speech rate, and tremor consistent with autonomic activation rather than the flattened-affect deviations emphasized in Example 1. Sleep onset latency, total sleep time, and nocturnal awakenings are logged contemporaneously through the smartphone interface.

3.6 Sex-specific dosage parameters. The dosage-guidance system described herein applies sex-specific parameters. In the present Example, in which the subject is a male of post-pubertal age, the system applies male-parameter dosing in which menstrual-cycle phase is not an input and in which dosing recommendations instead incorporate, by way of non-limiting example, a slower expected hepatic clearance of co-administered benzodiazepines in older male subjects, age-adjusted lean-body-mass scaling for the g/kg dose calculation, and a higher prior probability of comorbid mild sleep-disordered breathing as a modifier of the § 3.4(c) overnight target. The same algorithmic structure is applied with sex-specific parameters distinct from those used in female subjects as set forth in Examples 1 and 2, consistent with the disclosure that calculations involving subjects of either sex are not performed identically and that female subjects are not treated as scaled male subjects.

3.7 Hyperbolic deprescription ramp (Days 29-393). Beginning on Day 29, and only upon clinical determination by the prescribing clinician that taper of clonazepam is appropriate, clonazepam is reduced according to a hyperbolic taper schedule consistent with the benzodiazepine deprescription literature, in which proportional dose reductions decrease in absolute magnitude as the total daily dose decreases, so as to maintain approximately constant proportional reductions of GABA-A receptor occupancy across each taper step. By way of non-limiting example, clonazepam total daily dose is reduced by approximately 10% of the then-current dose every 14 to 28 days, with the inter-step interval lengthening as the dose decreases (e.g., 1.0→0.9→0.8→0.7→0.6→0.5→0.4→0.3→0.25→0.2→0.15→0.125→0.1→0.0625→0.03125→0 mg/day), with intermediate dose magnitudes achieved by liquid titration or by compounded micro-tablets. D-β-hydroxybutyric acid administration is continued at 36 g/day as in § 3.3, and the dosage-guidance system increases the time-averaged blood BHB floor during taper by, for example, advancing the 07:30 unit dose by 30-60 minutes ahead of the subject's habitual wake time so that ketotic coverage is established before the morning awakening window of § 3.4(a), and by adding a fourth unit dose of 6-9 g at approximately 04:00-05:00 during the most vulnerable taper steps (typically the final 25% of total dose reduction, where proportional receptor-occupancy changes per step are largest). Should the smartphone-based assessments register a STAI elevation of 10 or more points above the subject's stabilization-phase baseline sustained for more than 72 hours, a sleep-onset latency exceeding 60 minutes for more than three consecutive nights, voice-feature deviation flags indicative of sustained autonomic activation, or any reported new-onset perceptual disturbance, the guidance system flags the event for the prescribing clinician and recommends pausing further clonazepam reduction at the current dose for an additional taper-step interval and increasing D-β-hydroxybutyric acid time-averaged exposure pending clinical review.

3.8 Post-cessation continuation phase (Days 394 et seq.). Following complete cessation of clonazepam, administration of D-β-hydroxybutyric acid with vitamin B6 is continued at 0.25-0.5 g/kg/day for at least an additional 180 days, and in some embodiments at least 365 days, in recognition of the protracted post-acute withdrawal syndrome (PAWS) that is well documented following long-tenure benzodiazepine cessation and during which neural network destabilization, anxiety lability, and sleep architecture disruption may persist for months to years. The longer post-cessation continuation period of the present Example, relative to the 90-day default of Example 1 and the 90-day default of Example 2, reflects the longer duration over which excitatory-inhibitory rebalancing is expected following cessation of a long-half-life, long-tenure GABAergic agent.

3.9 Expected outcomes. It is expected that, relative to a comparable hyperbolic clonazepam taper conducted without D-β-hydroxybutyric acid administration:
(a) the subject maintains capillary blood BHB≥0.6 mmol/L during the morning awakening window, the mid-afternoon interdose window, and at least the first 4 hours of the overnight period throughout the stabilization, taper, and post-cessation continuation phases;
(b) STAI scores remain within 10 points of the subject's stabilization-phase baseline throughout the taper, and BDI-II scores remain within 5 points of that baseline;
(c) the number of clinically required pauses or reversals of taper steps is reduced relative to a comparable taper conducted without D-β-hydroxybutyric acid administration;
(d) sleep-onset latency, total sleep time, and number of nocturnal awakenings remain within the subject's stabilization-phase ranges, and no new-onset perceptual disturbances are reported;
(e) capillary blood glucose remains within the subject's pre-treatment range, indicating that the metabolic support is provided by exogenous ketone availability rather than by induced hypoglycemia;
(f) gastrointestinal tolerability and palatability-driven adherence are improved relative to administration of an equimolar dose of a D/L ketone salt, an equimolar dose of a ketone monoester, or an equimolar dose of 1,3-butanediol, by reason of the absence of counter-ion load, ester hydrolysis byproducts, and hepatic alcohol-to-ketone conversion intermediates, respectively; and
(g) post-acute symptoms during the § 3.8 continuation phase are reduced in frequency and magnitude relative to a comparable post-cessation interval conducted without D-β-hydroxybutyric acid administration.
3.10 Non-limiting nature and applicability to other benzodiazepines. The dose magnitudes, dose frequencies, intra-day timing, blood BHB targets, taper schedule, and post-cessation continuation duration set forth in this Example are illustrative and non-limiting. In other embodiments, unit doses range from 2 g to 40 g, daily totals range from 0.1 g/kg/day to 1.5 g/kg/day, dose frequencies range from twice daily to six times daily, blood BHB targets range from 0.6 mmol/L to 5.0 mmol/L, taper-step proportional reductions range from approximately 5% to approximately 25% of then-current dose, taper-step intervals range from 7 to 56 days, and post-cessation continuation extends from 90 days to 24 months or longer, without departing from the scope of the invention. The methods of this Example are further applicable, with appropriate dose and timing adjustments, to other benzodiazepines including without limitation alprazolam, lorazepam, diazepam, temazepam, and oxazepam, and to non-benzodiazepine GABAergic hypnotics including without limitation zolpidem (Ambien), zopiclone/eszopiclone, and zaleplon. In the case of benzodiazepines of short elimination half-life such as alprazolam (t½ approximately 11 hours) and lorazepam (t½ approximately 12 hours), the sustained-coverage timing strategy of § 3.3 is replaced or supplemented with a peri-dose-trough-bracketing timing strategy substantially as set forth in § 2.3 of Example 2, in which D-β-hydroxybutyric acid unit doses are administered within approximately 60 minutes prior to each scheduled benzodiazepine dose so as to maintain blood BHB at or above 0.3 mmol/L during the peri-dose pharmacokinetic trough of the benzodiazepine. In the case of benzodiazepines of intermediate or long half-life such as clonazepam, diazepam, and the active long-half-life metabolites thereof, the sustained-coverage timing strategy of § 3.3 is applied substantially as set forth above. Selection between the two timing strategies, or a hybrid thereof, is made by the dosage-guidance system based on the elimination half-life of the centrally acting pharmaceutical, the dosing frequency thereof, and the subject's logged symptom-timing data.

Example 4 Prophetic Co administration of Bioidentical D β Hydroxybutyric Acid with B Vitamins During Deprescription of ADHD medication in a Young Adult Male with ADHD, Anxiety, and Irritability

4.1 Subject. A 17 year old male subject of 75 kg body weight, having received a centrally acting stimulant medication for attention deficit/hyperactivity disorder ADHD at a stable maintenance dose for approximately 36 months, is enrolled in a supervised deprescription protocol. By way of non limiting example, the stimulant is an extended release mixed amphetamine salts preparation (Adderall XR) administered once daily at a dose of 30 mg each morning. Over the preceding year, the subject has developed clinically significant anxiety, irritability, and episodic anger outbursts temporally associated with stimulant use and with late day stimulant offset. Laboratory assessment obtained prior to enrollment demonstrates low normal or deficient levels of vitamin B2 riboflavin and vitamin B6 (pyridoxal 5 phosphate), with B9, B12, and homocysteine in the reference range. Baseline measures include: (a) Adult ADHD Self Report Scale ASRS, (b) a validated anxiety scale (e.g., GAD 7), (c) Beck Depression Inventory-II BDI II, (d) a brief irritability or anger scale, (e) capillary blood β hydroxybutyrate BHB less than 0.2 mmol/L on waking, and (f) capillary blood glucose between 80 and 100 mg/dL on waking. The subject has no contraindication to ketogenic intervention, no history of type 1 diabetes or inborn error of fatty acid oxidation, and no active substance use disorder.

4.2 Composition administered. Each unit dose comprises bioidentical D β hydroxybutyric acid in salt free, ester free, and alcohol free form together with B vitamin co factors configured to address the subject's documented low B2 and B6 levels. In a preferred embodiment, each unit dose contains 10 g of D β hydroxybutyric acid, optional 5 -25 mg vitamin B6 (preferably as pyridoxal 5 phosphate, alternatively as pyridoxine), optional 5-20 mg vitamin B2 riboflavin, optional 200-800 μg vitamin B9 (preferably as methylfolate), optional 250-1000 μg vitamin B12 (preferably as methylcobalamin), and optional 250-1500 mg betaine trimethylglycine TMG, formulated as a liquid concentrate suitable for dilution. Each unit dose is diluted in approximately 300-800 mL of cold water immediately prior to consumption to improve palatability and support adherence, consistent with other embodiments of the invention. In alternative embodiments, the same quantities of D BHB and B vitamins are provided in capsule or liquid drink form within a kit that further comprises printed or electronic instructions for use during stimulant treatment and deprescription.

4.3 Dosing regimen. The subject is administered the unit dose orally three times per day, corresponding to a total daily D β hydroxybutyric acid intake of approximately 30 g/day or about 0.4 g/kg/day for a 75 kg male, within the disclosed range of 0.25 g/kg/day to 1.0 g/kg/day. Unit doses are scheduled to provide neurometabolic support during (a) the morning interval in which Adderall XR reaches therapeutic concentration, (b) the late afternoon interval in which stimulant levels fall and anxiety/irritability typically emerge, and (c) the early night interval in which sleep onset difficulty and carry over agitation may occur. By way of non limiting example, D BHB unit doses are administered at approximately 0600 (approximately 60-90 minutes before breakfast and before Adderall XR ingestion), 1300 (mid day), and 2000 (approximately 2 hours before intended bedtime), while the subject limits intake to not more than three discrete meals per day, avoids caloric snacks between meals, and maintains total daily carbohydrate intake below approximately 100 g.

4.4 Pharmacokinetic targets and relation to anxiety/irritability windows. Following each oral dose, capillary blood BHB is expected to rise above approximately 0.6 mmol/L within about 10-20 minutes, reach peak concentrations of approximately 1.0-2.5 mmol/L within about 30-60 minutes, and remain at or above approximately 0.6 mmol/L for at least 2 hours post dose, consistent with the pharmacokinetic targets described in other prophetic examples. The three times daily regimen is configured such that: (a) morning D BHB exposure overlaps the onset of Adderall XR effect, buffering neural networks during the transition to pharmacologically increased catecholaminergic tone; (b) mid day and late afternoon D BHB exposure overlaps declining stimulant levels and the period in which rebound anxiety, irritability, and anger are expected to be most prominent; and (c) evening D BHB exposure supports sleep onset stability and overnight glymphatic network recovery, while avoiding peak BHB levels above approximately 5.0 mmol/L.

4.5 Stabilization phase Days 1-28. D β hydroxybutyric acid together with vitamins B2, B6, B9, B12, and betaine are administered as described in 4.3 while the Adderall XR dose is held constant at 30 mg each morning. The subject maintains the specified meal pattern and carbohydrate restriction, and uses a smartphone enabled system to log meals, capture daily capillary blood BHB (for example in the late afternoon, at least 2 hours after the mid day dose), and capture morning capillary blood glucose. ADHD symptoms (ASRS), anxiety (e.g., GAD 7), depression (BDI II), and irritability/anger scores are recorded at least weekly via the smartphone interface, and optional passive monitoring (voice tone, typing patterns, and device interaction metrics) is used to detect emerging changes in arousal, agitation, or mood instability relative to the subject's baseline. The software assisted guidance system processes diet, BHB, glucose, and symptom data to refine timing of D BHB unit doses within the day while maintaining the same total daily D BHB exposure.

4.6 Deprescription ramp Days 29-180. Beginning on approximately Day 29, the Adderall XR dose is reduced in accordance with the prescribing clinician's taper schedule, by way of non limiting example a 10-25% reduction in total daily stimulant dose every 21-30 days using successively lower strength extended release capsules and, where necessary, immediate release rescue doses to achieve a linear or hyperbolic taper profile. D β hydroxybutyric acid and B vitamin administration is continued at approximately 30 g D BHB per day as in 4.3, and the dosage guidance system adjusts D BHB dose timing and, where indicated, per dose magnitude in response to: (a) late afternoon BHB readings trending below 0.6 mmol/L during taper intervals, (b) clinically meaningful increases in anxiety, irritability/anger, or ASRS scores relative to the stabilized pre taper baseline, or (c) passive signal deviation flags suggesting increased agitation, impulsivity, or sleep disturbance. In certain embodiments, as the Adderall XR dose is reduced into the lower range, the system advances the timing of the evening D BHB dose by 30-60 minutes and may add an optional small pre sleep D BHB dose (for example 5 -7.5 g without additional B vitamins) to reinforce early night ketotic support while minimizing any risk of sleep fragmentation.

### 4.7 Post Cessation Continuation Phase Days 181-360.

Following complete cessation of Adderall XR, which under clinical supervision may occur between approximately Day 150 and Day 210, administration of D β hydroxybutyric acid with B vitamins is continued at a reduced total daily dose of approximately 0.25-0.5 g/kg/day for at least an additional 6 months. During this continuation phase, dosing frequency may be reduced to twice daily (for example, morning and early evening), while maintaining a pattern of limited meal frequency and reduced carbohydrate intake to support endogenous ketogenesis and continued gut barrier support. The software assisted system continues to track ASRS, anxiety, mood, and irritability scores, along with BHB and glucose values, and gradually steps down exogenous D BHB exposure as attentional function, anxiety, and anger related symptoms remain stable in the absence of stimulant medication.

4.8 Expected outcomes. Relative to a comparable Adderall XR deprescription conducted without D β hydroxybutyric acid and targeted B vitamin support, the present method is expected to reduce the intensity and duration of late day anxiety, irritability, and anger episodes during taper and post cessation, while maintaining ADHD symptom control within a clinically acceptable range. It is further expected that capillary blood BHB remains at or above approximately 0.6 mmol/L through a substantial portion of the late afternoon and early night intervals across the taper, that B2 and B6 status normalize into the mid reference range, that BDI II and anxiety scores remain near baseline, and that capillary blood glucose remains within the subject's pre treatment range, indicating that the neurometabolic benefits are achieved without hypoglycemia. Gastrointestinal tolerability and overall adherence are expected to be superior to racemic ketone salt, ketone ester, or 1,3 butanediol formulations by virtue of the bioidentical, salt free D BHB acid formulation described in the broader specification.

4.9 Non limiting nature. The specific subject profile, dose magnitudes, B vitamin selections, pharmacokinetic targets, taper steps, and continuation duration described in this Example are illustrative and non limiting, and may be varied within the broader ranges disclosed herein while remaining within the scope of the invention. In additional embodiments, the methods of this Example are applied to male or female subjects across adolescent and adult age ranges, to related stimulant formulations including lisdexamfetamine and methylphenidate, and to subjects with differing patterns of anxiety, irritability, or B vitamin status, with corresponding adjustments in D BHB and B vitamin dosing, software guided monitoring thresholds, and taper trajectory.

We claim:

1. A composition comprising: (a) bioidentical D-beta-hydroxybutyric acid in free acid form, wherein the D-beta-hydroxybutyric acid is the enantiomerically pure D-enantiomer of beta-hydroxybutyric acid substantially free of the L-enantiomer, and is salt-free, ester-free, and alcohol-free; (b) at least one B vitamin; wherein the composition is free of ketone salts, ketone esters, and 1,3-butanediol; and wherein the composition is formulated as an ingestible dosage form selected from the group consisting of a liquid concentrate and a powder.

2. The composition of claim 1, wherein the at least one B vitamin comprises vitamin B6.

3. The composition of claim 2, wherein the vitamin B6 is provided as pyridoxal-5-phosphate.

4. The composition of claim 1, wherein the at least one B vitamin further comprises one or more members selected from the group consisting of vitamin B1, vitamin B2, vitamin B9, vitamin B12, betaine, and combinations thereof.

5. The composition of claim 4, wherein the vitamin B9 is methylfolate and the vitamin B12 is methylcobalamin.

6. The composition of claim 1, wherein the composition is formulated as a unit dose comprising from 5 grams to 20 grams of the D-beta-hydroxybutyric acid, the unit dose being configured for dissolution in from 300 to 800 milliliters of water prior to oral administration, or as a powder unit dose enclosed in a sealed single-serving container.

7. The composition of claim 1, wherein the D-beta-hydroxybutyric acid, as the enantiomerically pure D-enantiomer in free acid form, acts on intestinal epithelial tight junction proteins to reduce paracellular leakiness and improve intestinal barrier integrity, and wherein the composition is formulated without counter-ion mineral load, ester hydrolysis byproducts, or hepatic alcohol-to-ketone conversion intermediates that would impede immediate bioavailability at the intestinal epithelium.

8. The composition of claim 1, wherein the composition is provided in a plurality of unit doses configured for administration according to a dosing regimen that accepts menstrual cycle phase as a timing input for female subjects, wherein dose timing or dose magnitude is adjusted during a peri-menstrual window to maintain blood beta-hydroxybutyrate concentration above 0.6 mmol/L, and wherein dosing parameters for male subjects are distinct from those for female subjects.

9. A kit comprising: (a) a plurality of unit-dose containers of bioidentical D-beta-hydroxybutyric acid in free acid form, wherein the D-beta-hydroxybutyric acid is the enantiomerically pure D-enantiomer of beta-hydroxybutyric acid substantially free of the L-enantiomer, and is salt-free, ester-free, and alcohol-free, and wherein the D-beta-hydroxybutyric acid is provided in a liquid concentrate form, a powder form, or both; and (b) instructions for use directing administration of the D-beta-hydroxybutyric acid and a neurologically acting pharmaceutical agent according to a three-phase deprescription protocol comprising a stabilization phase, a deprescription ramp phase concurrent with stepwise dose reduction of the neurologically acting pharmaceutical agent, and a post-tapering maintenance phase.

10. The kit of claim 9 additionally comprising (c) a plurality of pre-measured doses of at least one neurologically acting pharmaceutical agent, wherein the pre-measured doses are configured in stepwise decreasing amounts and are provided for concurrent administration under clinician supervision;

11. The kit of claim 10, wherein the neurologically acting pharmaceutical agent is selected from the group consisting of selective serotonin reuptake inhibitors, antiepileptic drugs, benzodiazepines, ADHD stimulants, and combinations thereof.

12. The kit of claim 9, wherein the unit-dose containers further contain at least one B vitamin and the at least one B vitamin comprises vitamin B6 provided as pyridoxal-5-phosphate.

13. The kit of claim 10, wherein the pre-measured doses of the neurologically acting pharmaceutical agent are configured according to a hyperbolic taper schedule in which proportional dose reductions decrease in absolute magnitude as the total daily dose of the neurologically acting pharmaceutical agent decreases, so as to maintain approximately constant proportional reductions in receptor occupancy across each taper step.

14. The kit of claim 9, wherein the stabilization phase spans at least 14 days of D-beta-hydroxybutyric acid administration prior to any dose reduction of the neurologically acting pharmaceutical agent, the deprescription ramp phase comprises stepwise dose reductions at intervals of from 7 to 56 days, and the post-tapering maintenance phase continues D-beta-hydroxybutyric acid administration for at least 90 days following cessation of the neurologically acting pharmaceutical agent.

15. The kit of claim 10, wherein the D-beta-hydroxybutyric acid is provided in powder form co-packaged with a pre-measured dose of the neurologically acting pharmaceutical agent in a single unit-dose container configured for combined oral administration.

16. The kit of claim 9, wherein the instructions for use further comprise sex-specific dosing parameters, wherein dosing parameters for female subjects incorporate menstrual cycle phase as a dosing variable, and dosing parameters for male subjects are distinct from dosing parameters for female subjects.

17. A system for neurometabolic deprescription support comprising: (a) a plurality of unit-dose containers of bioidentical D-beta-hydroxybutyric acid in free acid form, wherein the D-beta-hydroxybutyric acid is the enantiomerically pure D-enantiomer of beta-hydroxybutyric acid substantially free of the L-enantiomer, and is salt-free, ester-free, and alcohol-free; (b) a plurality of pre-measured doses of at least one neurologically acting pharmaceutical agent configured in stepwise decreasing amounts for concurrent administration under clinician supervision; (c) instructions for use directing administration according to a three-phase deprescription protocol comprising a stabilization phase, a deprescription ramp phase, and a post-tapering maintenance phase; and (d) a biomarker monitoring component comprising at least one of a blood beta-hydroxybutyrate test strip, a blood glucose test strip, a continuous ketone monitoring device, and a breath acetone analyzer, wherein the biomarker monitoring component is configured to measure blood or breath ketone levels in a subject during administration of the D-beta-hydroxybutyric acid according to the three-phase deprescription protocol.

18. The system of claim 17, further comprising a software-assisted guidance system configured to: (a) receive from a user device at least one of dietary intake data, medication dosing data, blood beta-hydroxybutyrate measurements, blood glucose measurements, and mood assessment data; and (b) output individualized guidance for at least one of timing and magnitude of D-beta-hydroxybutyric acid administration, dietary adjustments, and coordination with a clinician-supervised dose-reduction schedule for the neurologically acting pharmaceutical agent.

19. The system of claim 17, further comprising a glucose-ketone index calculation module configured to receive blood beta-hydroxybutyrate measurements and blood glucose measurements from the biomarker monitoring component, to calculate a glucose-ketone index value as the ratio of blood glucose concentration in mmol/L to blood beta-hydroxybutyrate concentration in mmol/L, and to output the glucose-ketone index value to a user device to guide at least one of the timing and magnitude of D-beta-hydroxybutyric acid administration during the three-phase deprescription protocol.

20. The system of claim 19, further comprising: (a) a software-assisted guidance system configured to receive from a user device at least one of dietary intake data, medication dosing data, blood beta-hydroxybutyrate measurements, blood glucose measurements, and mood assessment data, and to output individualized guidance for at least one of timing and magnitude of D-beta-hydroxybutyric acid administration, dietary adjustments, and coordination with a clinician-supervised dose-reduction schedule for the neurologically acting pharmaceutical agent; and (b) a remote laboratory data integration module configured to receive at least one of serum zonulin, aldosterone, HbA1c, homocysteine, vitamin B6, vitamin B9, and vitamin B12 laboratory values from an external laboratory data source, and to incorporate the received laboratory values into the individualized guidance output of the software-assisted guidance system.
